(19) United States
(12) Patent Application Publication
(10) Pub. No.: US 2024/0215927 A1
(43) Pub. Date: Jul. 4, 2024

(54) DEEP LEARNING SUPER-RESOLUTION TRAINING FOR ULTRA LOW-FIELD MAGNETIC RESONANCE IMAGING

(71) Applicant: Neuro42 Inc., San Francisco, CA (US)

(72) Inventor: Hung-Yu LIN, Walnut Creek, CA (US)

(73) Assignee: Neuro42 Inc., San Francisco, CA (US)

(21) Appl. No.: 18/147,556

(22) Filed: Dec. 28, 2022

(51) Int. Cl. A61B 5/00

(52) U.S. Cl. CPC A61B 5/742; A61B 5/7264

(57) ABSTRACT

The present disclosure provides systems and methods for deep learning super-resolution training and/or image generation for low-field and ultra-low field magnetic resonance imaging. In some aspects, a method includes obtaining a first image of a brain with a low-field strength magnetic resonance imaging system. The first image has a first resolution. The method further includes obtaining a deep learning brain model based on high-field strength images. The deep learning brain model can be configured to be applied by a neural network comprising a plurality of layers. The method further includes applying the deep learning brain model to the first image to generate a second image of the brain. The second image has a second resolution, and the second resolution is greater than the first resolution.

### BACKGROUND

The present disclosure relates to magnetic resonance imaging (MRI), medical imaging, medical intervention, and surgical intervention. MRI systems often include large and complex machines that generate significantly high magnetic fields and create significant constraints on the feasibility of certain surgical interventions. Restrictions can include limited physical access to the patient by a surgeon and/or a surgical robot and/or limitations on the usage of certain electrical and mechanical components in the vicinity of the MRI scanner. Such limitations are inherent in the underlying design of many existing systems and are difficult to overcome.

### SUMMARY

In one aspect, the present disclosure describes a method. The method can include obtaining a first image of a brain with a low-field strength magnetic resonance imaging system. The first image can include a first resolution. The method can further include obtaining a deep learning brain model based on high-field strength images. The deep learning brain model can be configured to be applied by a neural network comprising a plurality of layers. The method can further include applying the deep learning brain model to the first image to generate a second image of the brain. The second image can include a second resolution. The second resolution is greater than the first resolution.

In another aspect, the present disclosure describes a system. The system includes a processor and a memory. The memory can store machine-readable instructions. The processor can be configured to execute the machine-readable instructions. The machine-readable instructions, when executed, can implement a neural network. The neural network can be configured to obtain a high-field strength magnetic resonance model comprising a plurality of layers and receive data representative of a low-field image. The neural network can be further configured to convert the low-field image to a higher resolution image based on the high-field strength magnetic resonance model and output the higher resolution image.

### BRIEF DESCRIPTION OF THE DRAWINGS

The various aspects described herein, both as to organization and methods of operation, together with further objects and advantages thereof, may best be understood by reference to the following description, taken in conjunction with the accompanying drawings as follows.

FIG. 1 depicts components of a MRI scanning system including a dome-shaped housing for a magnetic array, the dome-shaped housing surrounding a region of interest therein and further depicting the dome-shaped housing positioned to receive at least a portion of the head of a patient reclined on the table into the region of interest, in accordance with at least one aspect of the present disclosure.

FIG. 1A depicts a patient's head positioned in the region of interest of the MRI scanning system of FIG. 1.

FIG. 2 is a perspective view of an alternative dome-shaped housing for a magnetic array for use with the MRI scanning system of FIG. 1, wherein access apertures are defined in the dome-shaped housing, in accordance with at least one aspect of the present disclosure.

FIG. 3 is a perspective view of an alternative dome-shaped housing for a magnetic array for use with the MRI scanning system of FIG. 1, wherein access apertures and an adjustable gap is defined in the dome-shaped housing, in accordance with at least one aspect of the present disclosure.

FIG. 4 depicts a dome-shaped housing for use with a MRI scanning system having an access aperture in the form of a centrally-defined hole, in accordance with at least one aspect of the present disclosure.

FIG. 5 is a cross-sectional view of the dome-shaped housing of FIG. 4, in accordance with at least one aspect of the present disclosure.

FIG. 6 depicts a control schematic for a MRI system, in accordance with at least one aspect of the present disclosure.

FIG. 7 is a flowchart depicting a method for obtaining imaging data from an MRI system, in accordance with at least one aspect of the present disclosure.

FIG. 8 depicts a MRI scanning system and a robotic system, in accordance with at least one aspect of the present disclosure.

FIG. 9 is a flowchart depicting a method for converting a low-resolution MRI image to a higher resolution MRI image, in accordance with at least one aspect of the present disclosure.

FIG. 10 is a flowchart depicting a method for converting a low-resolution MRI image to a super-resolution MRI image including implementing transfer learning to a high-field strength brain model based on a low-field strength dataset, in accordance with at least one aspect of the present disclosure.

FIG. 11 depicts arrays of MRI images including low-field, low-resolution MRI brain images and higher resolution MRI brain images converted from the low-field, low-resolution MRI brain images, in accordance with at least one aspect of the present disclosure.

FIG. 12 depicts arrays of MRI images including low-field, low-resolution MRI phantom images and super-resolution MRI phantom images converted from the low-field, low-resolution MRI phantom images, in accordance with at least one aspect of the present disclosure.

FIG. 13 is a block diagram of a system for converting a low-resolution MRI image to a higher resolution MRI image, in accordance with at least one aspect of the present disclosure.

Corresponding reference characters indicate corresponding parts throughout the several views. The exemplifications set out herein illustrate various disclosed embodiments, is one form, and such exemplifications are not to be construed as limiting the scope thereof in any manner.

### DETAILED DESCRIPTION

Applicant of the present application also owns the following patent applications that were filed on even date herewith and which are each herein incorporated by reference in their respective entireties:
U.S. Patent Application Attorney Docket No. 220407, titled MODULARIZED MULTI-PURPOSE MAGNETIC RESONANCE PHANTOM.
U.S. Patent Application Attorney Docket No. 220408, titled INTRACRANIAL RADIO FREQUENCY COIL FOR INTRAOPERATIVE MAGNETIC RESONANCE IMAGING.

Applicant of the present application also owns the following patent applications, which are each herein incorporated by reference in their respective entireties:
International Patent Application No. PCT/US2022/72143, titled NEURAL INTERVENTIONAL MAGNETIC RESONANCE IMAGING APPARATUS, filed May 5, 2022.
U.S. patent application Ser. No. 18/057,207, titled SYSTEM AND METHOD FOR REMOVING ELECTROMAGNETIC INTERFERENCE FROM LOW-FIELD MAGNETIC RESONANCE IMAGES, filed Nov. 19, 2022.

Before explaining various aspects of interventional magnetic resonance imaging devices in detail, it should be noted that the illustrative examples are not limited in application or use to the details of construction and arrangement of parts illustrated in the accompanying drawings and description. The illustrative examples may be implemented or incorporated in other aspects, variations and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects and/or examples.

Various aspects are directed to neural interventional magnetic resonance imaging (MRI) devices that allows for the integration of surgical intervention and guidance with an MRI. This includes granting physical access to the area around the patient as well as access to the patient's head with one or more access apertures. In addition, the neural interventional MRI device may allow for the usage of robotic guidance tools and/or traditional surgical implements. In various instances, a neural interventional MRI can be used intraoperatively to obtain scans of a patient's head and/or brain during a surgical intervention, such as a surgical procedure like a brain biopsy or neurosurgery.

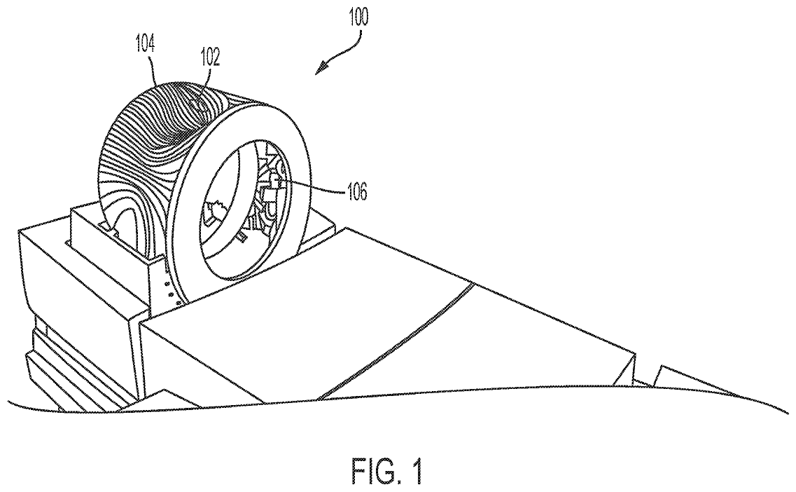

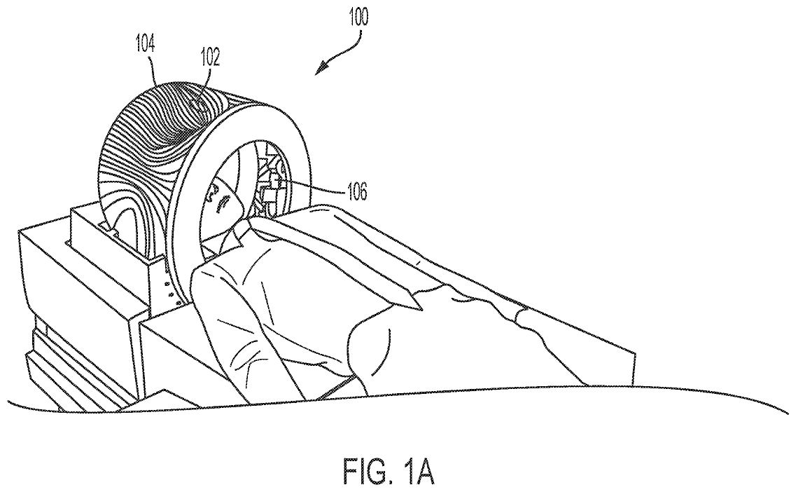

FIG. 1 depicts a MRI scanning system 100 that includes a dome-shaped housing 102 configured to receive a patient's head. The dome-shaped housing 102 can further include at least one access aperture configured to allow access to the patient's head to enable a neural intervention. A space within the dome-shaped housing 102 forms the region of interest for the MRI scanning system 100. Target tissue in the region of interest is subjected to magnetization fields/pulses, as further described herein, to obtain imaging data representative of the target tissue.

For example, referring to FIG. 1A, a patient can be positioned such that his/her head is positioned within the region of interest within the dome-shaped housing 102. The brain can be positioned entirely within the dome-shaped housing 102. In such instances, to facilitate intracranial interventions (e.g. neurosurgery) in concert with MR imaging, the dome-shaped housing 102 can include one or more apertures that provide access to the brain. Apertures can be spaced apart around the perimeter of the dome-shaped housing.

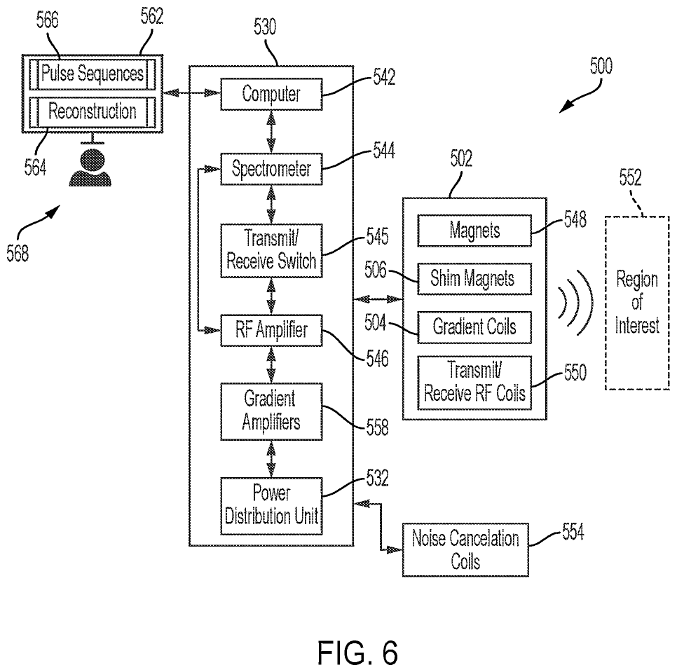

The MRI scanning system 100 can include an auxiliary cart (see, e.g. auxiliary cart 540 in FIG. 6) that houses certain conventional MRI electrical and electronic components, such as a computer, programmable logic controller, power distribution unit, and amplifiers, for example. The MRI scanning system 100 can also include a magnet cart that holds the dome-shaped housing 102, gradient coil(s), and/or a transmission coil, as further described herein. Additionally, the magnet cart can be attached to a receive coil in various instances. Referring primarily to FIG. 1, the dome-shaped housing 102 can further include RF transmission coils, gradient coils 104 (depicted on the exterior thereof), and shim magnets 106 (depicted on the interior thereof). Alternative configurations for the gradient coil(s) 104 and/or shim magnets 106 are also contemplated. In various instances, the shim magnets 106 can be adjustably positioned in a shim tray within the dome-shaped housing 102, which can allow a technician to granularly configure the magnetic flux density of the dome-shaped housing 102.

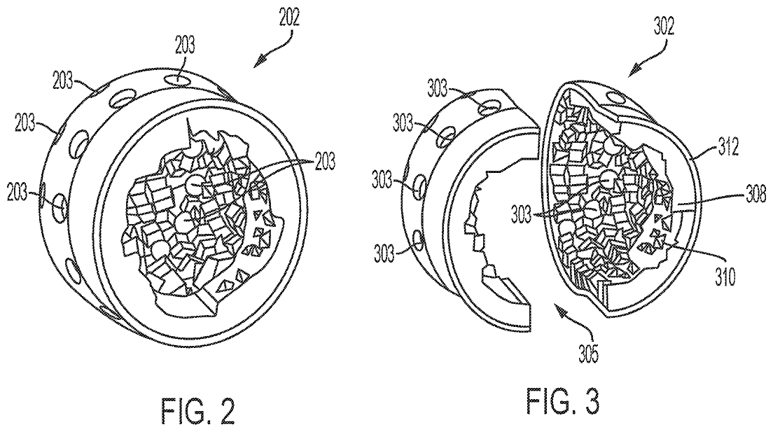

Various structural housings for receiving the patient's head and enabling neural interventions can be utilized with a MRI scanning system, such as the MRI scanning system 100. In one aspect, the MRI scanning system 100 may be outfitted with an alternative housing, such as a dome-shaped housing 202 (FIG. 2) or a two-part housing 302 (FIG. 3) configured to form a dome-shape. The dome-shaped housing 202 defines a plurality of access apertures 203; the two-part housing 302 also defines a plurality of access apertures 303 and further includes an adjustable gap 305 between the two parts of the housing.

In various instances, the housings 202 and 302 can include a bonding agent 308, such as an epoxy resin, for example, that holds a plurality of magnetic elements 310 in fixed positions. The plurality of magnetic elements 310 can be bonded to a structural housing 312, such as a plastic substrate, for example. In various aspects, the bonding agent 308 and structural housing 312 may be non-conductive or diamagnetic materials. Referring primarily to FIG. 3, the two-part housing 302 comprises two structural housings 312. In various aspect, a structural housing for receiving the patient's head can be formed from more than two sub-parts. The access apertures 303 in the structural housing 312 provide a passage directly to the patient's head and are not obstructed by the structural housing 312, bonding agent 308, or magnetic elements 310. The access apertures 303 can be positioned in an open space of the housing 302, for example.

There are many possible configurations of neural interventional MRI devices that can achieve improved access for surgical intervention. Many configurations build upon two main designs, commonly known as the Halbach cylinder and the Halbach dome described in the following article: Cooley et al. (e.g. Cooley, C. Z., Haskell, M. W., Cauley, S. F., Sappo, C., Lapierre, C. D., Ha, C. G., Stockmann, J. P., & Wald, L. L. (2018). Design of sparse Halbach magnet arrays for portable MRI using a genetic algorithm. IEEE transactions on magnetics, 54(1), 5100112. The article “Design of sparse Halbach magnet arrays for portable MRI using a genetic algorithm” by Cooley et al., published in IEEE transactions on magnetics, 54(1), 5100112 in 2018, is incorporated by reference herein in its entirety.

In various instances, a dome-shaped housing for an MRI scanning system, such as the system 100, for example, can include a Halbach dome defining a dome shape and configured based on several factors including main magnetic field B0 strength, field size, field homogeneity, device size, device weight, and access to the patient for neural intervention. In various aspects, the Halbach dome comprises an exterior radius and interior radius at the base of the dome. The Halbach dome may comprise an elongated cylindrical portion that extends from the base of the dome. In one aspect, the elongated cylindrical portion comprises the same exterior radius and interior radius as the base of the dome and continues from the base of the dome at a predetermined length, at a constant radius. In another aspect, the elongated cylindrical portion comprises a different exterior radius and interior radius than the base of the dome (see e.g. FIGS. 2 and 3). In such instances, the different exterior radius and interior radius of the elongated cylindrical portion can merge with the base radii in a transitional region.

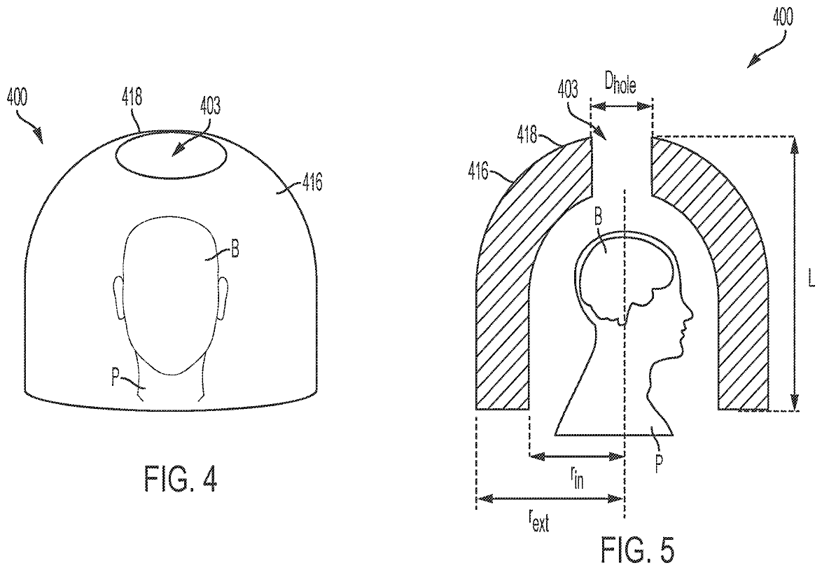

FIG. 4 illustrates an exemplary Halbach dome 400 for an MRI scanning system, such as the system 100, for example, which defines an access aperture in the form of a hole or access aperture 403, where the dome 400 is configured to receive a head and brain B of the patient P within the region of interest therein, and the access aperture 403 is configured to allow access to the patient P to enable neural intervention with a medical instrument and/or robotically-controlled surgical tool, in accordance with at least one aspect of the present disclosure. The Halbach dome 400 can be built with a single access aperture 403 at the top side 418 of the dome 400, which allows for access to the top of the skull while minimizing the impact to the magnetic field. Additionally or alternatively, the dome 300 can be configured with multiple access apertures around the structure 416 of the dome 400, as shown in FIGS. 2 and 3.

The diameter Dhole of the access aperture 403 may be small (e.g. about 2.54 cm) or very large (substantially the exterior rext diameter of the dome 400). As the access aperture 403 becomes larger, the dome 400 begins to resemble a Halbach cylinder, for example. The access aperture 403 is not limited to being at the apex of the dome 400. The access aperture 403 can be placed anywhere on the surface or structure 416 of the dome 400. In various instances, the entire dome 400 can be rotated so that the access aperture 403 can be co-located with a desired physical location on the patient P.

FIG. 5 depicts relative dimensions of the Halbach dome 400, including a diameter Dhole of the access aperture 403, a length L of the dome 400, and an exterior radius rext and an interior radius rin of the dome 400. The Halbach dome 400 comprises a plurality of magnetic elements that are configured in a Halbach array and make up a magnetic assembly. The plurality of magnetic elements may be enclosed by the exterior radius rext and interior radius rin in the structure 416 or housing thereof. In one aspect, example dimensions may be defined as: rin=19.3 cm; rext=23.6 cm; L=38.7 cm; and 2.54 cm≤D<19.3 cm.

Based on the above example dimensions, a Halbach dome 400 with an access aperture 403 may be configured with a magnetic flux density B0 of around 72 mT, and an overall mass of around 35 kg. It will be appreciated that the dimensions may be selected based on particular applications to achieve a desired magnetic flux density B0, total weight of the Halbach dome 400 and/or magnet cart, and geometry of the neural intervention access aperture 403.

In various aspects, the Halbach dome 400 may be configured to define multiple access apertures 403 placed around the structure 416 of the dome 400. These multiple access apertures 403 may be configured to allow for access to the patient's head and brain B using tools (e.g., surgical tools) and/or a surgical robot.

In various aspects, the access aperture 403 may be adjustable. The adjustable configuration may provide the ability for the access aperture 403 to be adjusted using either a motor, mechanical assist, or a hand powered system with a mechanical iris configuration, for example, to adjust the diameter Dhole of the access aperture 403. This would allow for configuration of the dome without an access aperture 403, conducting an imaging scan, and then adjusting the configuration of the dome 400 and mechanical iris thereof to include the access aperture 403 and, thus, to enable a surgical intervention therethrough.

Halbach domes and magnetic arrays thereof for facilitating neural interventions are further described in International Patent Application No. PCT/US2022/72143, titled NEURAL INTERVENTIONAL MAGNETIC RESONANCE IMAGING APPARATUS, filed May 5, 2022, which is incorporated by reference herein in its entirety.

Referring now to FIG. 6, a schematic for an MRI system 500 is shown. The MRI scanning system 100 (FIG. 1) and the various dome-shaped housings and magnetic arrays therefor, which are further described herein, for example, can be incorporated into the MRI system 500, for example. The MRI system 500 includes a housing 502, which can be similar in many aspects to the dome-shaped housings 102 (FIG. 1), 202 (FIG. 2), and/or 302 (FIG. 3), for example. The housing 502 is dome-shaped and configured to form a region of interest, or field of view, 552 therein. For example, the housing 502 can be configured to receive a patient's head in various aspects of the present disclosure.

The housing 502 includes a magnet assembly 548 having a plurality of magnets arranged therein (e.g. a Halbach array of magnets). In various aspect, the main magnetic field B0, generated by the magnetic assembly 548, extends into the field of view 552, which contains an object (e.g. the head of a patient) that is being imaged by the MRI system 500.

The MRI system 500 also includes RF transmit/receive coils 550. The RF transmit/receive coils 550 are combined into integrated transmission-reception (Tx/Rx) coils. In other instances, the RF transmission coil can be separate from the RF reception coil. For example, the RF transmission coil(s) can be incorporated into the housing 502 and the RF reception coil(s) can be positioned within the housing 502 to obtain imaging data.

The housing 502 also includes one or more gradient coils 504, which are configured to generate gradient fields to facilitate imaging of the object in the field of view 552 generated by the magnet assembly 548, e.g., enclosed by the dome-shaped housing and dome-shaped array of magnetic elements therein. Shim trays adapted to receive shim magnets 506 can also be incorporated into the housing 502.

During the imaging process, the main magnetic field B0 extends into the field of view 552. The direction of the effective magnetic field (B1) changes in response to the RF pulses and associated electromagnetic fields transmitted by the RF transmit/receive coils 550. For example, the RF transmit/receive coils 550 may be configured to selectively transmit RF signals or pulses to an object in the field of view 552, e.g. tissue of a patient's brain. These RF pulses may alter the effective magnetic field experienced by the spins in the sample tissue.

The housing 502 is in signal communication with an auxiliary cart 530, which is configured to provide power to the housing 502 and send/receive control signals to/from the housing 502. The auxiliary cart 530 includes a power distribution unit 532, a computer 542, a spectrometer 544, a transmit/receive switch 545, an RF amplifier 546, and gradient amplifiers 558. In various instances, the housing 502 can be in signal communication with multiple auxiliary carts and each cart can support one or more of the power distribution unit 532, the computer 542, the spectrometer 544, the transmit/receive switch 545, the RF amplifier 546, and/or the gradient amplifiers 558.

The computer 542 is in signal communication with a spectrometer 544 and is configured to send and receive signals between the computer 542 and the spectrometer 544. When the object in the field of view 552 is excited with RF pulses from the RF transmit/receive coils 550, the precession of the object results in an induced electric current, or MR current, which is detected by the RF transmit/receive coils 550 and sent to the RF preamplifier 556. The RF preamplifier 556 is configured to boost or amplify the excitation data signals and send them to the spectrometer 544. The spectrometer 544 is configured to send the excitation data to the computer 542 for storage, analysis, and image construction. The computer 542 is configured to combine multiple stored excitation data signals to create an image, for example. In various instances, the computer 542 is in signal communication with at least one database 562 that stores reconstruction algorithms 564 and/or pulse sequences 566. The computer 542 is configured to utilize the reconstruction algorithms to generate an MRI image 568.

From the spectrometer 544, signals can also be relayed to the RF transmit/receive coils 550 in the housing 502 via an RF power amplifier 546 and the transmit/receive switch 545 positioned between the spectrometer 544 and the RF power amplifier 546. From the spectrometer 544, signals can also be relayed to the gradient coils 560 in the housing 502 via a gradient power amplifier 558. For example, the RF power amplifier 546 is configured to amplify the signal and send it to RF transmission coils 560, and the gradient power amplifier 558 is configured to amplify the gradient coil signal and send it to the gradient coils 560.

In various instances, the MRI system 500 can include noise cancellation coils 554. For example, the auxiliary cart 530 and/or computer 542 can be in signal communication with noise cancellation coils 554. In other instances, the noise cancellation coils 554 can be optional. For example, certain MRI systems disclosed herein may not include supplemental/auxiliary RF coils for detecting and canceling electromagnetic interference, i.e. noise.

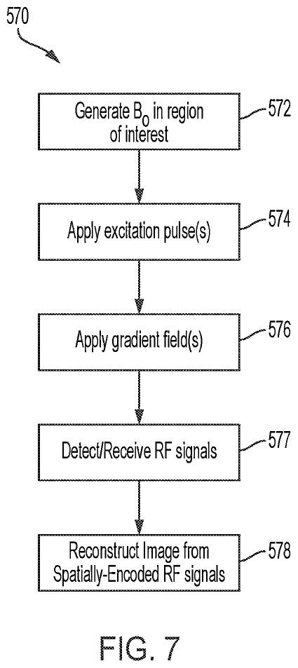

A flowchart depicting a process 570 for obtaining an MRI image is shown in FIG. 7. The flowchart can be implemented by the MRI system 500, for example. In various instances, at block 572, the target subject (e.g. a portion of a patient's anatomy), is positioned in a main magnetic field B0 in an interest of region (e.g. region of interest 552), such as within the dome-shaped housing of the various MRI scanners further described herein (e.g. magnet assembly 548). The main magnetic field B0 is configured to magnetically polarize the hydrogen protons (1H-protons) of the target subject (e.g. all organs and tissues) and is known as the net longitudinal magnetization M0. It is proportional to the proton density (PD) of the tissue and develops exponentially in time with a time constant known as the longitudinal relaxation time T1 of the tissue. T1 values of individual tissues depend on a number of factors including their microscopic structure, on the water and/or lipid content, and the strength of the polarizing magnetic field, for example. For these reasons, the T1 value of a given tissue sample is dependent on age and state of health.

At block 574, a time varying oscillatory magnetic field B1, i.e. an excitation pulse, is applied to the magnetically polarized target subject with a RF coil (e.g. RF transmit/receive coil 550). The carrier frequency of the pulsed B1 field is set to the resonance frequency of the 1H-proton, which causes the longitudinal magnetization to flip away from its equilibrium longitudinal direction resulting in a rotated magnetization vector, which in general can have transverse as well as longitudinal magnetization components, depending on the flip angle used. Common B1 pulses include an inversion pulse, or a 180-degree pulse, and a 90-degree pulse. A 180-degree pulse reverses the direction of the 1H-proton's magnetization in the longitudinal axis. A 90-degree pulse rotates the 1H-proton's magnetization by 90 degrees so that the magnetization is in the transverse plane. The MR signals are proportional to the transverse components of the magnetization and are time varying electrical currents that are detected with suitable RF coils. These MR signals decay exponentially in time with a time constant known as the transverse relaxation time T2, which is also dependent on the microscopic tissue structure, water/lipid content, and the strength of the magnetic field used, for example.

At block 576, the MR signals are spatially encoded by exposing the target subject to additional magnetic fields generated by gradient coils (e.g. gradient coils 560), which are known as the gradient fields. The gradient fields, which vary linearly in space, are applied for short periods of time in pulsed form and with spatial variations in each direction. The net result is the generation of a plurality of spatially encoded MR signals, which are detected at block 577, and which can be reconstructed to form MRI images depicting slices of the examination subject. A RF reception coil (e.g. RF transmit/receive coil 550) can be configured to detect the spatially-encoded RF signals. Slices may be oriented in the transverse, sagittal, coronal, or any oblique plane.

At block 578, the spatially encoded signals of each slice of the scanned region are digitized and spatially decoded mathematically with a computer reconstruction program (e.g. by computer 542) in order to generate images depicting the internal anatomy of the examination subject. In various instances, the reconstruction program can utilize an (inverse) Fourier transform to back-transforms the spatially-encoded data (k-space data) into geometrically decoded data.

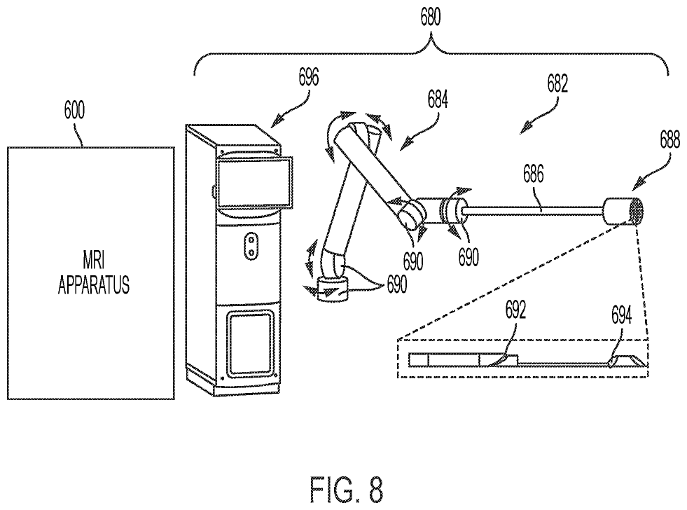

FIG. 8 depicts a graphical illustration of a robotic system 680 that may be used for neural intervention with an MRI scanning system 600. The robotic system 680 includes a computer system 696 and a surgical robot 682. The MRI scanning system 600 can be similar to the MRI system 500 and can include the dome-shaped housing and magnetic arrays having access apertures, as further described herein. For example, the MRI system 500 can include one or more access apertures defined in a Halbach array of magnets in the permanent magnet assembly to provide access to one or more anatomical parts of a patient being imaged during a medical procedure. In various instances, a robotic arm and/or tool of the surgical robot 682 is configured to extend through an access aperture in the permanent magnet assembly to reach a patient or target site. Each access aperture can provide access to the patient and/or surgical site. For example, in instances of multiple access apertures, the multiple access apertures can allow access from different directions and/or proximal locations.

In accordance with various embodiments, the robotic system 680 is configured to be placed outside the MRI system 600. As shown in FIG. 8, the robotic system 680 can include a robotic arm 684 that is configured for movements with one or more degrees of freedom. In accordance with various embodiments, the robotic arm 684 includes one or more mechanical arm portions, including a hollow shaft 686 and an end effector 688. The hollow shaft 686 and end effector 688 are configured to be moved, rotated, and/or swiveled through various ranges of motion via one or more motion controllers 690. The double-headed curved arrows in FIG. 8 signify exemplary rotational motions produced by the motion controllers 690 at the various joints in the robotic arm 684.

In accordance with various embodiments, the robotic arm 684 of the robotic system 682 is configured for accessing various anatomical parts of interest through or around the MRI scanning system 600. In accordance with various embodiments, the access aperture is designed to account for the size of the robotic arm 684. For example, the access aperture defines a circumference that is configured to accommodate the robotic arm 684, the hollow shaft 686, and the end effector 688 therethrough. In various instances, the robotic arm 684 is configured for accessing various anatomical parts of the patient from around a side of the magnetic imaging apparatus 600. The hollow shaft 686 and/or end effector 688 can be adapted to receive a robotic tool 692, such as a biopsy needle having a cutting edge 694 for collecting a biopsy sample from a patient, for example.

The reader will appreciate that the robotic system 682 can be used in combination with various dome-shaped and/or cylindrical magnetic housings further described herein. Moreover, the robotic system 682 and robotic tool 692 in FIG. 8 are exemplary. Alternative robotic systems can be utilized in connection with the various MRI systems disclosed herein. Moreover, handheld surgical instruments and/or additional imaging devices (e.g. an endoscope) and/or systems can also be utilized in connection with the various MRI systems disclosed herein.

In various aspects of the present disclosure, the MRI systems described herein can comprise low field MRI (LF-MRI) systems. In such instances, the main magnetic field B0 generated by the permanent magnet assembly can be between 0.1 T and 1.0 T, for example. In other instances, the MRI systems described herein can comprise ultra-low field MRI (ULF-MRI) systems. In such instances, the main magnetic field B0 generated by the permanent magnet assembly can be between 0.03 T and 0.1 T, for example.

Higher magnetic fields, such as magnetic fields above 1.0 T, for example, can preclude the use of certain electrical and mechanical components in the vicinity of the MRI scanner. For example, the existence of surgical instruments and/or surgical robot components comprising metal, especially ferrous metals, can be dangerous in the vicinity of higher magnetic fields because such tools can be drawn toward the source of magnetization. Moreover, higher magnetic fields often require specifically-designed rooms with additional precautions and shielding to limit magnetic interference. Despite the limitations on high-field MRI systems, low-field and ultra-low field MRI systems present various challenges to the acquisition of high quality images with sufficient resolution for achieving the desired imaging objectives.

LF- and ULF-MRI systems generally define an overall magnetic field homogeneity that is relatively poor in comparison to higher field MRI systems. For example, a dome-shaped housing for an array of magnets, as further described herein, can comprise a Halbach array of permanent magnets, which generate a magnetic field B0 having a homogeneity between 1,000 ppm and 10,000 ppm in the region of interest in various aspects of the present disclosure.

Images obtained with LF- or ULF-MRI systems can be referred to as low-field images. Images obtained with high-field MRI system can be referred to as high-field images. Training datasets for machine learning that are comprised of low-field images can be referred to as low-field datasets. Training datasets for machine learning that are comprised of high-field images can be referred to as high-field datasets.

In various instances, the intrinsic MRI signal is proportional to field strength. Consequently, the signal-to-noise (SNR) associated with a LF- and ULF-MRI system can theoretically be up to twenty times lower than the SNR for a high-field MRI system. For example, for a 70 mT MRI system, the SNR can be approximately 5% the SNR for a 1.5 T MRI system.

To improve spatial resolution, MRI systems can increase the number of k-space lines. However, an increase in the number k-space lines can further reduce the SNR and/or increase the scan time required for each scan. In various LF and ULF-MRI systems and methods, it may not be desirable to further decrease the SNR and/or increase the scan time.

In various instances, deep learning can be employed to obtain higher spatial resolution without a SNR decrease and/or without increasing the scan time. Deep learning techniques are further described in the 2018 article “Deep Back-Projection Networks for Super-Resolution” by Muhammad Haris, Greg Shakhnarovich and Norimichi Ukita (Haris, Muhammad & Shakhnarovich, Greg & Ukita, Norimichi. (2018), for example. Deep Back-Projection Networks for Super-Resolution. 1664-1673. 10.1109/CVPR.2018.00179), which is incorporated by reference herein in its entirety.

Such deep learning techniques typically utilize a high quality MRI training dataset. The high quality MRI training dataset can include a large quantity of MRI images. For example, to ensure statistical significance and robust analysis, MRI training datasets employed for deep learning studies can include at least hundreds of 3D MRI volumes, with each of the 3D MRI volumes including at least, 20 2D slices. Thus, a typical high quality MRI training dataset used for deep-learning training can include over 2000 2D slices. Increasing the size of the dataset may increase the variance in the data, and therefore may ensure a higher statistical significance and generate a more accurate model. However, in various instances, collecting a large quantity of high-resolution images with a particular type of MRI system may not be practical. For example, it may not be practical to obtain a high quality training dataset of images obtained via non-clinical and/or pre-FDA-approved MRI systems (e.g. certain low-field MRI systems) that are in an early stage of research and development.

Instead of relying exclusively on a high quality MRI training dataset based on low-field images, a low-field, low-resolution image can be converted to a higher resolution image using a pre-trained model where the model is trained with a high-field dataset, e.g. with a high quality training dataset based on high-field images. For example, the pre-trained model can be based on images obtained from a high-field MRI system, such as a clinically-available 1.5 T MRI system. In various instances, a pre-trained model trained with images from a 1.5 T MRI system can be publically available, for example.

In such instances, a low-field, low-resolution image can be converted to a higher resolution image, such as a high-resolution or super-resolution image, for example, without sacrificing SNR and/or without requiring a longer scan time. As used herein, a “high-resolution” image can sometimes refer to an image with a spatial resolution equal to or higher than 1.5 mm×1.5 mm. As used herein, a “low-resolution” image can sometimes refer to an image with a spatial resolution equal to or lower than 3.0×3.0 mm. As used herein, a “normal-resolution” image can sometimes refer to an image with a spatial resolution between 1.5 mm×1.5 mm and 3.0×3.0 mm. Generally, a higher image resolution can correlate with a smaller pixel size (e.g., an image with a spatial resolution of 1.5 mm×1.5 mm is higher resolution than an image with a spatial resolution of 3.0 mm×3.0 mm). As used herein, a “super-resolution” image can refer to an image that has been generated from an original image data set, wherein the super-resolution image has a higher resolution that the resolution of the original image data set. For example, a super-resolution image can be a high-resolution image generated from a low-resolution image dataset. As another example, a super-resolution image can be a normal-resolution image generated from a low-resolution dataset. As yet another example, a super-resolution image can be a ultra-high-resolution image generated from a high-resolution image dataset.

In various instances, portions of the pre-trained model, such as a subset of the neural layers, for example, can be re-trained based on a low-field dataset that is smaller than the high-field dataset utilized to generate the pre-trained model.

In such instances, the pre-trained model can be customized and/or fine-tuned to adjust for differences between a high-field model, i.e. based on high-field images, and a low-field model, i.e. based on low-field images. In various instances, re-training of a pre-trained model, or portions/layer(s) thereof, is referred to as transfer learning.

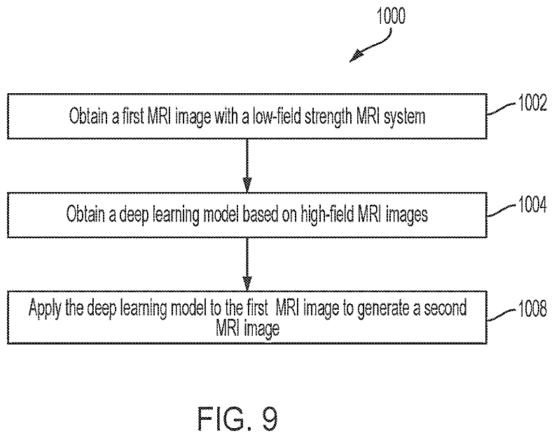

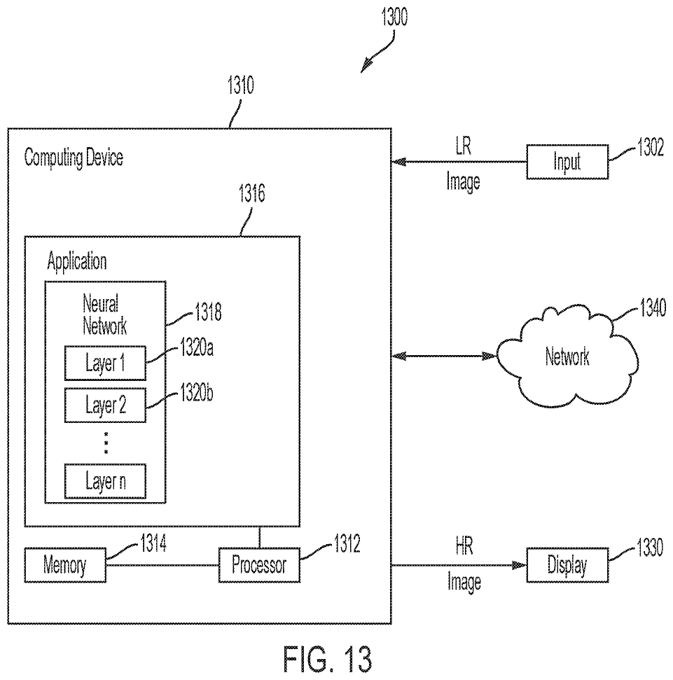

Referring now to FIG. 9, a flowchart 1000 is shown. The flowchart 1000 depicts a method for converting a low-resolution MRI image to a higher resolution MRI image by applying a deep learning model to the low-resolution MRI image. As further described herein, the flowchart 1000 utilizes a high-field deep learning model to improve the resolution of a low-field image. In various instances, the flowchart 1000 and/or portions thereof can be implemented by a computing device, such as the computing device 1310 (FIG. 13). The first MRI image can be accessible to the system 1300 of FIG. 13, for example.

At block 1002, a first MRI image is obtained. The first MRI image can be obtained from a LF- or ULF-MRI system. In various instances, the first MRI image at block 1002 can be obtained from the MRI scanning system 100 (FIG. 1) and/or MRI system 500 (FIG. 6), for example. The first MRI image can be an image of a brain, or a portion thereof, for example.

In various instances, the first MRI image obtained at block 1002 can be obtained from a LF- or ULF-MRI system. In certain instances, the MRI system used to obtain the first MRI image at block 1002 can be configured to generate a low magnetic field strength of less than 1 T in the field of view. In various instances, the low magnetic field strength can be less than 100 mT, and, in certain instances, can be about 70 mT, for example.

In certain instances, the first MRI image can be stored in a memory, such as the memory 1314 of the computing device 1310 (FIG. 13), for example, and/or can be transmitted from a remote computing device, such as a remote network and/or cloud storage device 1340 (FIG. 13), for example.

The resolution of the first MRI image can depend on the field strength of the MRI system, for example. The resolution of the first MRI image can be a low-resolution image having a spatial resolution equal to or lower than 3.0 mm×3.0, such as, for example, a low-resolution image having a spatial resolution in a range of 3.0 mm×3.0 to 5.0 mm×5.0 mm. The reader will appreciate that the foregoing exemplary ranges are based on obtaining the first MRI image at block 1002 with a LF- or ULF-MRI system, for example.

At block 1004, a deep learning model can be obtained. The deep learning model can be stored in a memory, such as the memory 1314 of the computing device 1310 (FIG. 13), for example, and/or can be transmitted from a remote computing device, such as a remote network and/or cloud storage device 1340 (FIG. 13), for example. The deep learning model can be accessible to the system 1300 of FIG. 13, for example. The deep learning model is based on high-field images.

In various instances, the deep learning brain model can be based on MRI images obtained with MRI systems configured to generate a high magnetic field of more than 1 T in the field of view. In various instances, the high magnetic field strength can be about 1.5 T or 3 T in the field of view, for example.

In various instances, the deep learning model can be a pre-trained model. In other instances, the deep learning model can be obtained by training a model based on a high-field dataset comprising high-field, high-resolution images and high-field, low-resolution images. For example, the high-field, low-resolution images can be augmented based on the high-field high-resolution images. Upon comparing the augmented high-field, low-resolution images and the corresponding high-field high-resolution images, the model can be trained based on the augmentation and comparison.

In various aspects, the deep learning model is implemented via a neural network. For example, the deep learning model can include multiple neural layers forming a neural network. Neural networks are computational models used in machine learning. Neural networks are generally made up of nodes organized in neural layers. The nodes are configured to perform a function on provided input to produce some output value. A neural network requires a training period to learn the parameters, i.e., weights, used to map the input to a desired output. The mapping occurs via the function. Thus, the weights are weights for the mapping function of the neural network.

At block 1008, the deep learning model can be applied to the first MRI image to generate a second MRI image. The resolution of the second MRI image can be greater than the resolution of the first MRI image. In some instances, the second MRI image can have a resolution that is improved by a factor of two compared with the first image. For example, where the first image has a spatial resolution of 3.0 mm×3.0 mm, the second MRI image can have a spatial resolution of 1.5 mm×1.5 mm. In other instances, the second MRI image can have a resolution that is improved by greater than a factor of two compared with the first image.

In various instances, the amount of resolution improvement achieved by generating the second MRI image can be controlled based on the training of the deep learning model. For example, in some instances, the amount of resolution improvement achieved can be optimized based on the feasibility of performing clinical analysis on a second MRI image with having a given resolution and the computational cost of achieving the given resolution. In some instances, configuring the deep learning model to generate the second MRI image to have a resolution that is improved by a factor of two compared with the first image enables the second MRI image to be suitable for clinical analysis while also minimizing the computational cost required to generate the second MRI image. In some instances, configuring the deep learning model to generate the second MRI image to have a resolution that is improved by a factor of two compared to the first MRI image enables quasi-real-time analysis of the second MRI image, for example, because of the minimized computational cost required to generate the second MRI image.

In various instances, upon generating the second MRI image at block 1008, the flowchart can be configured to output the second MRI image. The second MRI image can be provided to a clinician and/or patient, for example. In various instances, the second MRI image can be displayed on a graphical user interface, such as a computer screen, for example.

As further described herein, converting a low-resolution MRI image to a higher resolution MRI image by applying a deep learning model as set forth in the flowchart 1000 can further include implementing a transfer learning technique to fine-tune the deep learning model. Transfer learning can be utilized to re-train at least one layer of the deep learning model based on a low-field dataset. In various instances, a subset of the neural layers of the neural network implementing the deep learning model can be re-trained. Re-training at least one neural layer of the deep learning model can include accessing a low-field dataset comprising low-field, high-resolution images and low-field, low-resolution images. The low-field, low-resolution images can be augmented based on the low-field, high-resolution images. Upon comparing the augmented low-field, low-resolution images and the corresponding low-field, high-resolution images, at least one neural layer can be re-trained based on the augmentation and comparison.

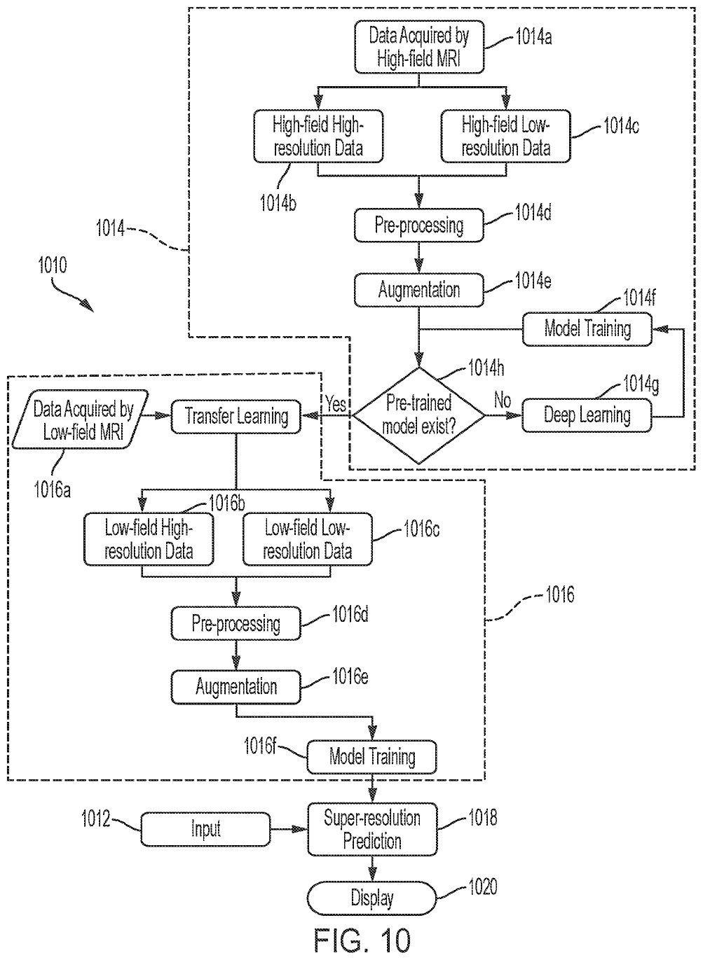

Referring now to FIG. 10, another flowchart 1010 for converting a low-resolution MRI image to a higher resolution MRI image is depicted. The flowchart 1010 includes a model training subroutine 1014 based on a high-field dataset 1014a and a transfer learning subroutine 1016 based on a low-field dataset 1016a. At block 1018, the transfer-learned trained model is applied to an input from block 1012 to generate a super-resolution prediction. The super-resolution prediction from block 1018 can be output to a display at block 1020 In various instances, the flowchart 1010 and/or portions thereof can be implemented by a computing device, such as the computing device 1310 (FIG. 13).

The model training subroutine 1014 includes utilizing a high-field dataset 1014a acquired by one or more high-field MRI systems. The high-field dataset 1014a includes high-field, high-resolution imaging data 1014b and high-field, low-resolution imaging data 1014c. In various instances, the high-field dataset 1014a can be based on MRI images obtained with MRI systems configured to generate a high magnetic field of more than 1 T in the field of view. In various instances, the high-field dataset 1014a can be based on MRI images obtained with MRI systems configured to generate a high magnetic field strength of about 1.5 T or 3 T in the field of view, for example.

At block 1014d, the high-field dataset 1014a is pre-processed. In some instances, the pre-processing can include distortion correction (e.g., correction of geometry distortion resulting from field inhomogeneity and/or gradient non-linearity), spatial normalization (e.g., spatial normalization to compensate for image intensity resulting from low-frequency intensity non-uniformity), and/or head masking (e.g. head masking to remove irrelevant artifacts from outside of the head). Then, at block 1014e, the high-field dataset 1014a is augmented. For example, the high-field, low-resolution imaging data 1014c can be augmented based on the high-field, high-resolution imaging data 1014b. In some instances, the augmentation can include image cropping, image rotation, image flipping, image filtering, image translation, image shearing, and/or image scaling. Upon comparing the augmented high-field low-resolution imaging data and the corresponding high-field high-resolution imaging data, the model can be trained at block 1014f. In various instances, model training at block 1014f can be iterative with the application of deep learning techniques at block 1014g. Deep learning techniques can include progressive upsampling and/or iterative upsampling and downsampling techniques. In one aspect of the present disclosure, the deep learning techniques applied at block 1014f can include deep back-projection networks as described in the 2018 article “Deep Back-Projection Networks for Super-Resolution” by Muhammad Haris, Greg Shakhnarovich and Norimichi Ukita for example, which is incorporated by reference herein in its entirety.

At block 1014h, the model can be evaluated to determine if a pre-trained model exists. Upon completion of the model training subroutine 1014, the flowchart 1010 can proceed to apply the transfer learning subroutine 1016.

In other aspects of the present disclosure, a pre-trained model can be obtained and provided to the computing device 1310 (FIG. 13). For example, the pre-trained model can be publically available. In certain instances, the pre-trained model can be downloaded from a remote network and/or cloud storage device, such as the network 1340 (FIG. 13), for example.

The pre-trained model from the model training subroutine 1014 is provided to the transfer learning subroutine 1016. The transfer learning subroutine 1016 includes utilizing a low-field dataset 1016a acquired by one or more low-field MRI systems. The low-field dataset 1016a includes low-field, high-resolution imaging data 1016b and low-field, low-resolution imaging data 1016c. In various instances, the low-field dataset 1016a can be based on MRI images obtained with MRI systems configured to generate a low magnetic field of less than 1 T in the field of view. In various instances, the low-field dataset 1016a can be based on MRI images obtained with MRI systems configured to generate a low magnetic field of about 100 mT or 70 mT in the field of view, for example.

At block 1016d, the low-field dataset 1016a is pre-processed. Then, at block 1014e, the low-field dataset 1016b is augmented. For example, the low-field low-resolution imaging data can be augmented based on the low-field high-resolution imaging data. Upon comparing the augmented low-field low-resolution imaging data 1016c and the corresponding low-field high-resolution imaging data 1016b, one or more layers of the model from the model training subroutine 1014 can be re-trained at block 1016f.

The model training subroutine 1014 and the transfer learning subroutine 1016 can be stored in a memory, such as the memory 1314 of the computing device 1310 (FIG. 13), for example, and/or can be transmitted from a remote computing device, such as a remote network and/or cloud storage device 1340, for example.

In various aspects, the deep learning model from the model training subroutine 1014 is implemented and/or applied to input via a neural network. For example, the deep learning model can be implemented via a neural network including multiple neural layers. A subset of the neural network can be re-trained with the transfer learning subroutine 1016. For example, less than ten percent or less than five percent of the neural layers can be re-trained with the transfer learning subroutine 1016. In various instances, four to ten neural layers can be re-trained with the transfer learning subroutine 1016. In still other instances, a single neural layer, such as the final layer in the neural network, for example, can be re-trained with the transfer learning subroutine 1016.

At block 1018, a super-resolution prediction is computed. For example, block 1018 can receive an input from block 1012 and apply the transfer-learned trained model from the model training subroutine 1014 and the transfer learning subroutine 1016 to the input. Block 1012 can be similar in many aspects to block 1002 (FIG. 9), for example. For example, the input can comprise imaging data that corresponds to a low-field MRI image.

In various instances, the super-resolution prediction can be output to a display at block 1020, such as the display 1330 (FIG. 13), for example. The output at block 1020 can be displayed on a graphical user interface, such as a computer screen, for example. The super-resolution prediction can include a MRI image having improved spatial resolution in comparison to the MRI image corresponding to the input provided at block 1012. In some instances, the super-resolution prediction output to a display at block 1020 can have a resolution that is improved by a factor of two for each of the three dimensional axes. For example, the input received from block 1020 can comprise imaging data having a spatial resolution of 3 mm×3 mm×6 mm and the super-resolution prediction output to a display at block 1020 can have a spatial resolution of 1.5 mm×1.5 mm×3.0 mm. Thus, the image can be improved from a low-resolution MRI image (e.g., 3 mm×3 mm) to a high-resolution MRI image (e.g., 1.5 mm×1.5 mm).

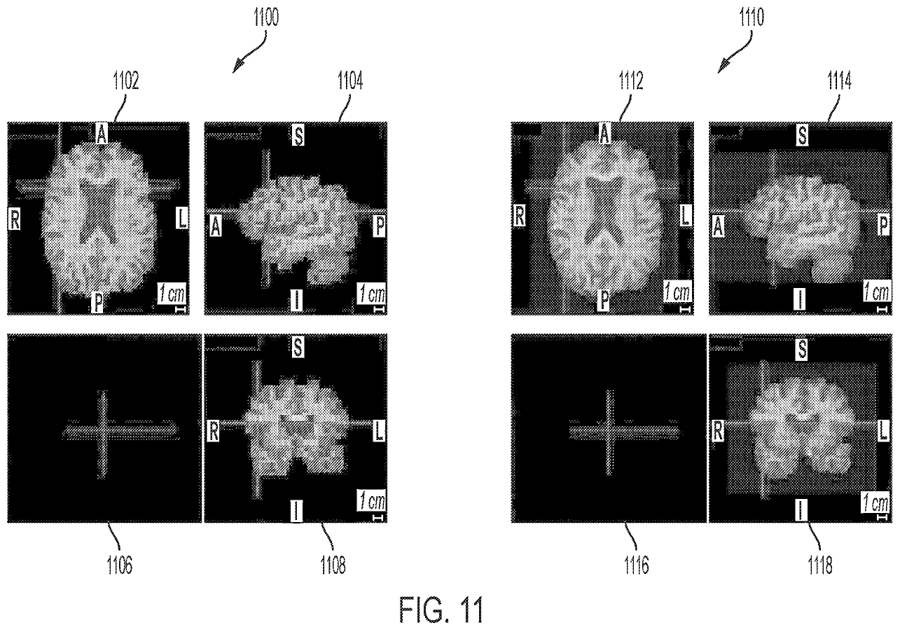

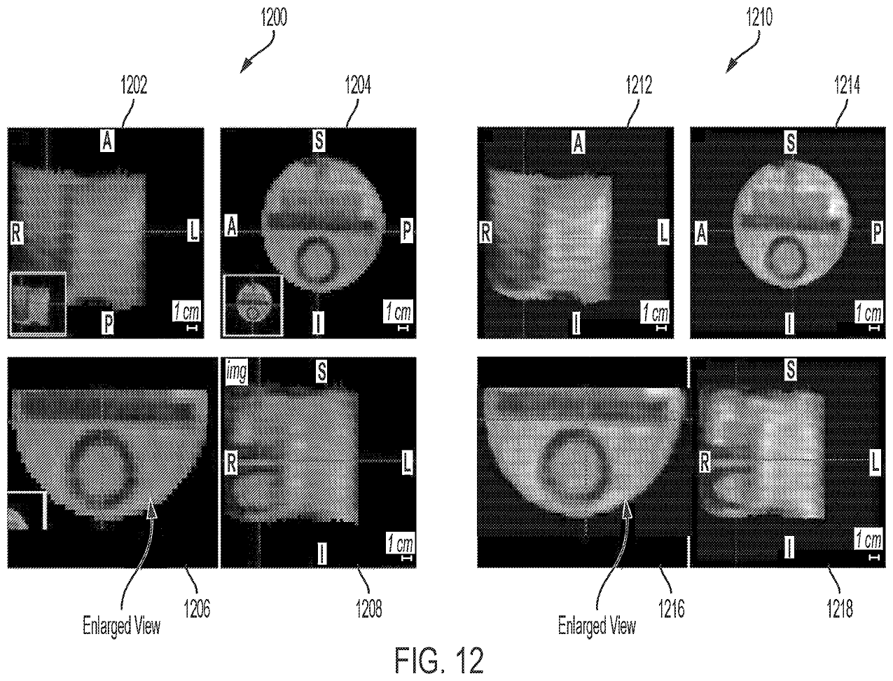

Exemplary MRI images are shown in FIGS. 11 and 12. FIG. 11 depicts a first array of MRI brain images 1100 including images 1102, 1104, 1106, and 1108 obtained in different imaging planes through the brain. FIG. 11 further depicts a second array of corresponding brain images 1110 including images 1112, 1114, 1116, and 1108, respectively. The first array of MRI brain images 1100 are lower resolution than the second array of MRI brain images 1110, which were reconstructed into high-resolution images without sacrificing SNR or scan time efficiency utilizing a pre-trained deep learning model based on high-field MRI data.

FIG. 12 depicts a first array of MRI phantom images 1200 including images 1202, 1204, 1206, and 1208 in different imaging planes through the phantom. FIG. 12 further depicts a second array of corresponding phantom images 1210 including images 1212, 1214, 1216, and 1208, respectively. The first array of MRI phantom images 1200 are lower resolution than the second array of MRI phantom images 1210, which were reconstructed into super-resolution images without sacrificing SNR or scan time efficiency utilizing a pre-trained deep learning model based on high-field MRI data.

Referring now to FIG. 13, a system 1300 for converting a low-resolution MRI image to a higher resolution MRI image is shown. The system 1300 includes a computing device 1310 having a memory 1314, a processor 1312, and an application 1316. The computing device 1310 may communicate with one or more other computing devices over a network 1340. The computing device 1310 may be implemented as a server, a desktop computer, a laptop computer and/or a mobile device, such as a tablet device or mobile phone device, for example. In various instances, the computing device 1310 may be representative of multiple computing devices in communication with one another, such as multiple servers in communication with each another.

The processor 1312 may represent two or more processors on the computing device 1310 executing in parallel and utilizing corresponding instructions stored using the memory 1314. The memory 1314 represents a non-transitory computer-readable storage medium. The memory 1314 may represent one or more different types of memory utilized by the computing device 1310, for example. In addition to storing instructions, which allow the processor 1312 to implement the application 1316 and computer-readable instructions stored in the memory 1314, the memory 1314 may be used to store data, such imaging data, pre-trained models, algorithms, and/or subroutines thereof, for example.

The application 1316 may be accessed directly by a user of the computing device 1310. In other implementations, the application 1316 may be running on the computing device 1310 as a component of a cloud network where a user accesses the application 1316 from another computing device over a network, such as the network 1340. The application 1316 enables a user to convert a low-resolution MRI image to a higher resolution MRI image. The application 1316 can also include additional functionality, such as noise reduction and/or further editing and/or manipulation of the images.

The application 1316 includes a neural network 1318 comprising a plurality of layers 1320a, 1320b, etc. The neural network 1318 can be configured to convert the low-resolution MRI image to a higher resolution MRI image. For example, the neural network 1318 can be trained and/or a pre-trained neural network can be provided to the application 1316. The neural network 1318 is configured to apply a deep learning model to an input (e.g. input imaging data representative of a low-field, low-resolution MRI image) to generate an output (e.g. output imaging data representative of a low-field, high-resolution MRI image). As further described herein, the deep learning model can be based on a high-field model based on a high-field dataset or a high-field model updated via transfer learning based on a smaller low-field dataset. Various methods for converting a low-resolution image to a higher-resolution image are further described herein.

The system 1300 also includes an input device 1302, which can comprise a user interface, for example. The user interface can includes additional elements and components, such as other tools used for image editing and image manipulation and graphic design for use as part of the application 1316, for example.

In various instances, the input device 1302 can be incorporated into the computing device 1310. In other instances, the input device 1302 can be separate from the computing device 1310. In either event, the input device 1302 is configured to receive inputs related to the imaging data, pre-trained models, algorithms, and/or subroutines thereof that are accessed by the processor to convert a low-resolution MRI image (e.g. provided via the input device 1302 to the computing device 1310) to a higher resolution MRI image (e.g. output via the display 1330 from the computing device 1310). The input device 1302 may access images from the memory 1314 and/or from other storages locations either locally on computing device 1310 or remotely on other computing devices accessed across the network 1340.

In various instances, upon receiving input from the input device 1302, the processor 1312 of the computing device 1310 can implement the application 1316 to apply the neural network 1318 and layers 1320a, 1320b, etc. thereof to the input imaging data. For example, the machine-readable instructions stored in the memory 1314 can be run the application 1316 via the processor 1312 to convert a low-resolution MRI image to a higher resolution MRI image in accordance with the flowcharts 1000 (FIG. 9) and 1100 (FIG. 10), for example.

### EXAMPLES

Various additional aspects of the subject matter described herein are set out in the following numbered examples:

Example 1: A method, comprising: obtaining a first image of a brain with a low-field strength magnetic resonance imaging system, wherein the first image comprises a first resolution; obtaining a deep learning brain model based on high-field strength images, wherein the deep learning brain model is configured to be applied by a neural network comprising a plurality of layers; and applying the deep learning brain model to the first image to generate a second image of the brain, wherein the second image comprises a second resolution, and wherein the second resolution is greater than the first resolution.

Example 2: The method of Example 1, wherein obtaining the deep learning brain model based on high-field strength images comprises obtaining a pre-trained model.

Example 3: The method of Example 1, wherein obtaining the deep learning brain model based on high-field strength images comprises: accessing a high-field dataset comprising high-field strength high-resolution images and high-field strength low-resolution images; augmenting the high-field strength low-resolution images based on the high-field strength high-resolution images; and training the deep learning brain model based on the augmented high-field strength low-resolution images.

Example 4: The method of any one of Examples 1-3, further comprising performing transfer learning to fine-tune the deep learning brain model for the first image of the brain.

Example 5: The method of any one of Examples 1-3, further comprising re-training at least one layer of the deep learning brain model with a low-field dataset.

Example 6: The method of any one of Examples 1-3, further comprising re-training a subset of the layers of the deep learning brain model with a low-field dataset.

Example 7: The method of any one of Examples 1-3, further comprising: accessing a low-field dataset comprising low-field strength high-resolution images and low-field strength low-resolution images; augmenting the low-field strength low-resolution images based on the low-field strength high-resolution images; and re-training at least one layer of the deep learning brain model based on the augmented low-field strength low-resolution images.

Example 8: The method of any one of Example 1-7, further comprising outputting the second image of the brain to a display.

Example 9: The method of any one of Examples 1-7, further comprising displaying the second image of the brain.

Example 10. The method of any one of Examples 1-9, wherein obtaining the first image of the brain with the low-field strength magnetic resonance imaging system comprises generating a low magnetic field strength of less than 100 mT, and wherein the deep learning brain model is based on magnetic resonance imagining images obtained with a high magnetic field strength of more than 1 T.

Example 11: A system, comprising: a processor; and a memory storing machine-readable instructions, wherein the processor is configured to execute the machine-readable instructions, and wherein the machine-readable instructions, when executed, implement a neural network configured to: obtain a high-field strength magnetic resonance model comprising a plurality of layers; receive data representative of a low-field image; convert the low-field image to a higher resolution image based on the high-field strength magnetic resonance model; and output the higher resolution image.

Example 12: The system of Example 11, wherein the high-field strength magnetic resonance model comprises a pre-trained deep learning model.

Example 13: The system of Example 12, wherein the pre-trained deep learning model is trained with a high-field dataset comprising high-field strength high-resolution images and high-field strength low-resolution images.

Example 14: The system of Example 11, wherein the neural network is further configured to: obtain a high-field dataset comprising high-field strength high-resolution images and high-field strength low-resolution images; augment the high-field strength low-resolution images based on the high-field strength high-resolution images; and train the high-field strength magnetic resonance model based on the augmented high-field strength low-resolution images.

Example 15: The system of any one of Examples 11-14, wherein the neural network is further configured to re-train at least one layer of the high-field strength magnetic resonance model with a low-field dataset.

Example 16: The system of any one of Examples 11-14, wherein the neural network is further configured to: obtain a low-field dataset comprising low-field strength high-resolution images and low-field strength low-resolution images; augment the low-field strength low-resolution images based on the low-field strength high-resolution images; and re-train at least one layer of the high-field strength magnetic resonance model based on the augmented low-field strength low-resolution images.

Example 17: The system of Example 16, wherein the high-field dataset is larger than the low-field dataset.

Example 18: The system of any one of Examples 11-14, wherein the high-field strength magnetic resonance model is trained with high-field images obtained at a high magnetic field strength exceeding 1 T.

Example 19: The system of Example 18, wherein the low-field image is obtained at a low magnetic field strength of less than 0.3 T.

Example 20: The system of Example 18, wherein the low-field image is obtained at a low magnetic field strength of less than 100 mT.

Though various aspects disclosed herein are directed to brain imaging and/or neurological interventions, the reader will appreciate that the various systems and methods disclosed herein can be used to image other portions of a patient's anatomy and/or different structures in various instances.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a control circuit computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A method, comprising:
obtaining a first image of a brain with a low-field strength magnetic resonance imaging system, wherein the first image comprises a first resolution;
obtaining a deep learning brain model based on high-field strength images, wherein the deep learning brain model is configured to be applied by a neural network comprising a plurality of layers; and
applying the deep learning brain model to the first image to generate a second image of the brain, wherein the second image comprises a second resolution, and wherein the second resolution is greater than the first resolution.

2. The method of claim 1, wherein obtaining the deep learning brain model based on high-field strength images comprises obtaining a pre-trained model.

3. The method of claim 1, wherein obtaining the deep learning brain model based on high-field strength images comprises:
accessing a high-field dataset comprising high-field strength high-resolution images and high-field strength low-resolution images;
augmenting the high-field strength low-resolution images based on the high-field strength high-resolution images; and
training the deep learning brain model based on the augmented high-field strength low-resolution images.

4. The method of claim 3, further comprising performing transfer learning to fine-tune the deep learning brain model for the first image of the brain.

5. The method of claim 3, further comprising re-training at least one layer of the deep learning brain model with a low-field dataset.

6. The method of claim 3, further comprising re-training a subset of the layers of the deep learning brain model with a low-field dataset.

7. The method of claim 3, further comprising:
accessing a low-field dataset comprising low-field strength high-resolution images and low-field strength low-resolution images;
augmenting the low-field strength low-resolution images based on the low-field strength high-resolution images; and
re-training at least one layer of the deep learning brain model based on the augmented low-field strength low-resolution images.

8. The method of claim 3, further comprising outputting the second image of the brain to a display.

9. The method of claim 3, further comprising displaying the second image of the brain.

10. The method of claim 3, wherein obtaining the first image of the brain with the low-field strength magnetic resonance imaging system comprises generating a low magnetic field strength of less than 100 mT, and wherein the deep learning brain model is based on magnetic resonance imagining images obtained with a high magnetic field strength of more than 1 T.

11. A system, comprising:
a processor; and
a memory storing machine-readable instructions, wherein the processor is configured to execute the machine-readable instructions, and wherein the machine-readable instructions, when executed, implement a neural network configured to:
obtain a high-field strength magnetic resonance model comprising a plurality of layers;
receive data representative of a low-field image;
convert the low-field image to a higher resolution image based on the high-field strength magnetic resonance model; and
output the higher resolution image.

12. The system of claim 11, wherein the high-field strength magnetic resonance model comprises a pre-trained deep learning model.

13. The system of claim 12, wherein the pre-trained deep learning model is trained with a high-field dataset comprising high-field strength high-resolution images and high-field strength low-resolution images.

14. The system of claim 11, wherein the neural network is further configured to:
obtain a high-field dataset comprising high-field strength high-resolution images and high-field strength low-resolution images;
augment the high-field strength low-resolution images based on the high-field strength high-resolution images; and
train the high-field strength magnetic resonance model based on the augmented high-field strength low-resolution images.

15. The system of claim 14, wherein the neural network is further configured to re-train at least one layer of the high-field strength magnetic resonance model with a low-field dataset.

16. The system of claim 14, wherein the neural network is further configured to:
obtain a low-field dataset comprising low-field strength high-resolution images and low-field strength low-resolution images;
augment the low-field strength low-resolution images based on the low-field strength high-resolution images; and
re-train at least one layer of the high-field strength magnetic resonance model based on the augmented low-field strength low-resolution images.

17. The system of claim 16, wherein the high-field dataset is larger than the low-field dataset.

18. The system of claim 14, wherein the high-field strength magnetic resonance model is trained with high-field images obtained at a high magnetic field strength exceeding 1 T.

19. The system of claim 18, wherein the low-field image is obtained at a low magnetic field strength of less than 0.3 T.

20. The system of claim 18, wherein the low-field image is obtained at a low magnetic field strength of less than 100 mT.
